(19) United States
(12) Patent Application Publication
(10) Pub. No.: US 2012/0016309 A1
(43) Pub. Date: Jan. 19, 2012

(54) MICROARRAY DEVICE

(76) Inventors: Peter Nicholas Binks, Sandringham (AU); Michelle Marie Critchley, Caulfield North (AU); Robert Alexander Irving, Pakenham (AU); Colin William Pouton, Alphington (AU); Paul James White, Pascoe Vale South (AU)

(21) Appl. No.: 13/214,549

(22) Filed: Aug. 22, 2011

Related U.S. Application Data
(63) Continuation of application No. 12/020,079, filed on Jan. 25, 2008.
Application No. 12/020,079 is a continuation-in-part of application No. PCT/AU2006/001039, filed on Jul. 25, 2006.

(30) Foreign Application Priority Data
Jul. 25, 2005 (AU) 2005903918

(51) Int. Cl. A61M 5/00

(52) U.S. Cl. 604/173; 604/272

(57) ABSTRACT

A device is provided which is suitable for delivering at least one nanoparticle(s) to a subject. The device can be used to deliver a variety of nanoparticles, for example, therapeutic agents, directly through the outer layers of the skin without passing completely through the epidermis of the subject. Thus the device can be used to deliver therapeutic agents to a predetermined depth and avoid disturbing the pain receptors in the skin. Thus the device can be used to deliver agents, including therapeutic agents, in a non-invasive manner. A method of fabricating devices with associated nanoparticles is also provided.

### CROSS-REFERENCE TO RELATED APPLICATIONS

The present application is a continuation-in-part of International Application No. PCT/AU2006/001039, filed on Jul. 25, 2006, published as WO 2007/012114 on Feb. 1, 2007, and claiming priority to Australian Provisional Patent Application No 2005903918 filed on Jul. 25, 2005.

The foregoing applications, and each document cited or referenced in each of the present and foregoing applications, including during the prosecution of each of the foregoing applications (“application and article cited documents”), and any manufacturer's instructions or catalogues for any products cited or mentioned in each of the foregoing applications and articles and in any of the application and article cited documents, are hereby incorporated herein by reference. Furthermore, all documents cited in this text, and all documents cited or reference in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

### FIELD OF THE INVENTION

The present invention relates to methods and devices for delivery of nanoparticles. In particular, the present invention relates to microneedles and microneedle arrays suitable for delivering nanoparticles.

### BACKGROUND OF THE INVENTION

There has been an increase in interest in methods for the efficacious delivery of agents to organisms, including the delivery of therapeutic agents such as drugs. The delivery of agents to organisms is complicated by the inability of many molecules to permeate biological barriers. Biological barriers for which it is desirable to deliver molecules across include the skin (or parts thereof); the blood-brain barrier; mucosal tissue (e.g., oral, nasal, ocular, vaginal, urethral, gastrointestinal, respiratory); blood vessels; lymphatic vessels; or cell membranes (e.g., for the introduction of material into the interior of a cell or cells).

Traditional delivery methods such as oral administration are not suitable for all types of drugs as many drugs are destroyed in the digestive track or immediately absorbed by the liver. Administration intravenously via hypodermic needles is also considered too invasive and results in potentially undesirable spike concentrations of the delivered drug. Moreover, traditional delivery methods are often not useful for efficient targeting of the drug delivery.

One approach for delivery of drugs through the skin is through the use of transdermal patches. A transdermal patch can provide significantly greater effective blood levels of a beneficial drug because the drug is not delivered in spike concentrations as is the case with hypodermic injection and most oral administration. In addition, drugs administered via transdermal patches are not subjected to the harsh environment of the digestive tract.

Transdermal patches are currently available for a number of drugs. Commercially available examples of transdermal patches include scopolamine for the prevention of motion sickness, nicotine for aid in smoking cessation, nitroglycerin for the treatment of coronary angina pain, and estrogen for hormonal replacement. Generally, these systems have drug reservoirs sandwiched between an impervious backing and a membrane face which controls the steady state rate of drug delivery. Such patches rely on the ability of the drug to diffuse through the outer most layer of the skin, the stratum corneum, and eventually into the circulatory system of the subject. The stratum corneum is a complex structure of compacted keratinized cell remnants having a thickness of about 10-30 μm and forms an effective barrier to prevent both the inward and outward passage of most substances. The degree of diffusion through the stratum corneum depends on the porosity of the skin, the size and polarity of the drug molecules, and the concentration gradient across the stratum corneum. These factors generally limit this mode of delivery to a very small number of useful drugs with very small molecules or unique electrical characteristics.

One common method for increasing the porosity of the skin is by forming micropores or cuts through the stratum corneum. By penetrating the stratum corneum and delivering the drug to the skin in or below the stratum corneum, many drugs can be effectively administered. The devices for penetrating the stratum corneum generally include a plurality of micro sized needles or blades having a length to penetrate the stratum corneum without passing completely through the epidermis. Examples of these devices are disclosed in U.S. Pat. No. 5,879,326 to Godshall et al., U.S. Pat. No. 5,250,023 to Lee et al and U.S. Pat. No. 6,334,856. However, the efficacy of these methods for enhancing transdermal delivery has been limited, as after the micropores have been formed, the drug needs to be separately administered to the treated skin.

Moreover, these devices are usually made from silicon or other metals using etching methods. For example, U.S. Pat. No. 6,312,612 to Sherman et al. describes a method of forming a microneedle array using Micro-Electro-Mechanical Systems (MEMS) technology and standard microfabrication techniques. Although partially effective, the resulting microneedle devices are relatively expensive to manufacture and difficult to produce in large numbers. Moreover, these arrangements have limited applicability to the delivery of a very limited range of molecules.

### SUMMARY OF THE INVENTION

According to one aspect, the present invention provides a device suitable for delivering at least one nanoparticle comprising

a microneedle having at least one nanoparticle associated with at least part of a surface of the microneedle and/or at least part of the fabric of the microneedle.

The size of the nanoparticle(s) may be in the range between about 1 nm to about 1000 nm. Preferably, the size of the nanoparticle may be between about 50 nm to about 500 nm.

Preferably the device has at least two microneedles. The microneedles may be arranged in a non-patterned arrangement or other such configuration. In other implementations, the microneedles may be arranged in at least one array.

Preferably the nanoparticle(s) may be associated with at least a part of the external surface of the microneedle.

Preferably the nanoparticle(s) may be associated with pores on the surface of the microneedles.

In some implementations, the nanoparticle(s) may be associated with at least a part of the fabric of the microneedle.

The pore(s), cavities or the like, may be of two or more shapes, cross sections selected from the group comprising circular, elongated, square, triangular, etc.

In other implementations, the nanoparticle(s) may be associated with internal pores in the fabric of the microneedle.

Preferably the association may comprise covalent bonding or non-covalent interactions. The non-covalent interactions may be selected from one or more of the group comprising ionic bonds, hydrophobic interactions, hydrogen bonds, Van der Waals forces or Dipole-dipole bonds.

Preferably the association is via a covalent bond to a functional group on the microneedle.

Preferably the functional group(s) may be selected from the group comprising COOR, CONR₂, NH₂, SH, and OH, where R comprises a H; organic or inorganic chain.

The microneedle(s) may be fabricated from a porous or non-porous material selected from the group comprising metals, natural or synthetic polymers, glasses, ceramics, or combinations of two or more thereof.

With this implementation, the polymer may be selected from the group comprising: polyglycolic acid/polylactic acid, polycaprolactone, polyhydroxybutarate valerate, polyorthoester, and polyethylenoxide/polybutylene terepthalate, polyurethane, silicone polymers, and polyethylene terephthalate, polyamine plus dextran sulfate trilayer, high-molecular-weight poly-L-lactic acid, fibrin, methylmethacrylate (MMA) (hydrophobic, 70 mol %) and 2-hydroxyethyl methacrylate (HEMA) (hydrophilic 30 mol %), elastomeric poly(ester-amide)(co-PEA) polymers, polyetheretherketone (Peek-Optima), biocompatible thermoplastic polymer, conducting polymers, polystyrene or combinations of two or more thereof.

The microneedles may include a layer or coating on at least a part of the surface of the microneedle(s) of an electrically conductive material.

Preferably the electrically conductive material may be selected from the group comprising conducting polymers; conducting composite materials; doped polymers, conducting metallic materials or combinations of two or more thereof.

The conducting polymer may be selected from the group comprising substituted or unsubstituted polymers comprising polyaniline; polypyrrole; polysilicones; poly(3,4-ethylenedioxythiophene); polymer doped with carbon nanotubes; polymer doped with metal nanoparticles, or combinations of two or more thereof.

Preferably the thickness of the layer or coating may be between about 20 nm to about 20 μm.

The electrically conductive material may be layered or coated on the microneedle(s) by electrodeposition.

At least one nanoparticle may be contained in the electrically conductive material.

Preferably the nanoparticle(s) may be delivered to an organism and the microneedle(s) maybe fabricated from a biocompatible material, the microneedle(s) may also be non-biodegradable.

The microneedle may be solid.

The microneedle may have nanosized pores or cavities on its surface.

The nanoparticle(s) may be an active agent.

In another implementation, the nanoparticle(s) may be a carrier for an agent.

Preferably the nanoparticle maybe associated with an active agent.

The active agent(s) may be associated with the nanoparticle(s) by covalent bonding or non-covalent interactions.

The non-covalent interactions may be selected from any one or more of the group comprising ionic bonds, hydrophobic interactions, hydrogen bonds, Van der Waals forces or Dipole-dipole bonds.

The nanoparticle may encapsulate the active agent.

In another implementation, the active agent may be incorporated into the nanoparticle(s).

Preferably the nanoparticle(s) may be fabricated from a material selected the group comprising metals, semiconductors, inorganic or organic polymers, magnetic colloidal materials, or combinations of two or more thereof.

The metal may be selected from the group comprising gold, silver, nickel, copper, titanium, platinum, palladium and their oxides or combinations of two or more thereof.

The polymer may be selected from the group comprising a conducting polymer; a hydrogel; agarose; polyglycolic acid/polylactic acid; polycaprolactone; polyhydroxybutarate valerate; polyorthoester; polyethylenoxide/polybutylene terepthalate; polyurethane; polymeric silicon compounds; polyethylene terephthalate; polyamine plus dextran sulfate trilayer; high-molecular-weight poly-L-lactic acid; fibrin; copolymers of methylmethacrylate (MMA) and 2-hydroxyethyl methacrylate (HEMA), elastomeric poly(ester-amide)(co-PEA) polymers; n-butyl cyanoacrylate; polyetheretherketone (Peek-Optima); polystyrene or combinations of two or more thereof.

Preferably the active agent may be a biological agent. With this implementation, the biological agent may be a therapeutic and/or a diagnostic agent.

Preferably the therapeutic agent may be selected from the group comprising whole micro-organisms, viruses, virus like particles, peptides, proteins, carbohydrates, nucleic acid molecules, an oligonucleotide or a DNA or RNA fragment(s), lipids, organic molecules, biologically active inorganic molecules or combinations of two or more thereof.

Preferably the therapeutic agent may be a vaccine.

The vaccine may be selected from the group comprising a vector containing a nucleic acid, oligonucleotide, gene for expression as a vaccine or combinations of two or more thereof.

Preferably the vaccine may be selected from proteins or peptides as vaccines for diseases selected from the group comprising Johnes disease, liver fluke, bovine mastitis, meningococcal disease.

The vaccine may comprise a Johnes disease peptide. With this implementation, the peptide may be selected from the group comprising:

| | |
|---|---|
| NVESQPGGQPNT; | (SEQ ID NO: 1) |
| QYTDHHSSLLGP; | (SEQ ID NO: 2) |
| LYRPSDSSLAGP; | (SEQ ID NO: 3) |

and/or their variants.

The vaccine may comprise a bovine mastitis disease peptides. With this implementation, the peptide may be selected from the group comprising:

(SEQ ID NO: 4)
MKKWFLILMLLGIFGCATQPSKVAAITGYDSDYYARYIDPDENKITF
AINVDGFVEGSNQEILIRGIHHVLTDQNQKIVTKAELLDAIRHQMVL
LQLDYSYELVDFAPDAQLLTQDRRLLFANQNFEESVSLEDTIQEYLL
KGHVILRKRVEEPITHPTETANIEYKVQFATKDGEFHPLPIFVDYGL
EKHIGEKLTSDEFRKIAEEKLLQLYPDYMIDQKEYTIIKHNSLGQPR
YYSYQDHFSYEIQDRQRIMAKDPKSGKELGETQSIDNVFEKYLITKK
SYKP;
(SEQ ID NO: 5)
ILIRGIHHVL;
(SEQ ID NO: 6)
IRHQMVLLQL;

and/or their variants.

The vaccine may comprise a Meningococcal disease peptide. With this implementation, the peptide may be selected from the group comprising:

(SEQ ID NO: 7)
GRGPYVQADLAYAYEHITHDYP
(SEQ ID NO: 8)
STVSDYFRNIRTHSIHPRVSVGYDFGGWRIAADYARYRKWNDNKYSV;

and/or their variants.

The vaccine may comprise a Hepatitis C virus. With this implementation, the peptide may be selected from the group comprising:

| | |
|---|---|
| QDVKFPGGGVYLLPRRGPRL; | (SEQ ID NO: 9) |
| RRGPRLGVRATRKTSERSQPRGRRQ; | (SEQ ID NO: 10) |
| PGYPWPLYGNEGCGWAGWLLSPRGS; | (SEQ ID NO: 11) |

and/or their variants.

The diagnostic agent may be a detectable agent. Preferably the detectable agent is used in an assay.

The outer diameter of the microneedle(s) may be between about 1 μm and about 100 μm.

The length of the microneedle(s) may be between about 20 μm and 1 mm. Preferably the length of the microneedle(s) may be between about 20 μm and 250 μm. Preferably the microneedle(s) may be adapted to provide an insertion depth of less than about 100 to 150 μm.

Preferably the shape of the microneedle(s) tip may be selected from the group comprising square, circular, oval, cross needle, triangular, chevron, jagged chevron, half moon or diamond shaped.

In one implementation, the entire microneedle may be fabricated of nanoparticles.

According to another aspect, the present invention provides a method for fabricating a device for delivering nanoparticles, the device comprising an array of microneedles and at least one nanoparticle associated with at least part of a surface of the microneedle, the method comprising the steps of:
(i) lining at least a part of the surface of a microneedle array mould with the nanoparticles;
(ii) moulding the microneedles;
   wherein after demoulding, the nanoparticles are associated with the surface of the microneedles.

In yet another aspect, the present invention provides a method for fabricating a device for delivering nanoparticles, the device comprising an array of microneedles and at least one nanoparticle associated with the pores on the surface of the microneedle, the method comprising the steps of
i) inducing porosity on at least a part of the surface of the microneedles;
ii) associating the nanoparticles with at least a part of the pores.

Preferably the step of inducing a porosity on the surface of the microneedles comprises the steps of:
i) selective leaching of micro or nanoparticles incorporated into the microneedle surface;
ii) physical, chemical or electrochemical treatment of the surface of the microneedles.

In yet a further aspect, the present invention provides a method for fabricating a device for delivering nanoparticles, the device comprising an array of microneedles and at least one nanoparticle associated with at least part of the fabric of the microneedle, the method comprising the steps of:

moulding the microneedles in the presence of the nanoparticles;

wherein after demoulding, the nanoparticles are associated with at least part of the fabric of the microneedles.

In another further aspect, the present invention provides a method for fabricating a device for delivering nanoparticles, the device comprising an array of microneedles and at least one nanoparticle associated with at least a part of the external surface of the microneedle, the method comprising the steps of:
i) functionalizing at least a part of the external surface of the microneedles with functional groups;
ii) binding the nanoparticles to the introduced functional groups.

Preferably the functionalizing step may be selected from the group comprising oxidation, reduction, substitution, crosslinking, plasma, heat treatment or combinations of two or more thereof.

Preferably the introduced functional group(s) may be selected from the group comprising COOR, CONR₂, NH₂, SH, and OH, where R comprises a H or an organic or inorganic chain.

The methods of the invention may include the step of coating at least a part of the microneedles with an electrically conductive material.

Preferably the electrically conductive material may be selected from the group comprising conducting polymer; conducting composite material; doped polymer, conducting metallic materials or composites thereof.

Preferably the conducting polymer may be selected from the group of substituted or unsubstituted polymers comprising polyaniline; polypyrrole; polysilicone; poly(3,4-ethylenedioxythiophene); polymer doped with metal nanoparticles; or polymer doped with carbon nanotubes.

In yet a further aspect, the present invention provides a device suitable for delivering at least one agent comprising

a microneedle fabricated from an electrically conductive polymer and/or electrically conductive polymer composite, the microneedle having at least one agent associated with at least part of a surface of the microneedle and/or at least of part of the fabric of the microneedle.

In yet a further aspect, the present invention provides a device suitable for delivering at least one agent comprising

a microneedle fabricated from an electrically conductive material, the microneedle having at least one agent associated with at least part of a surface of the microneedle and/or at least of part of the fabric of the microneedle.

The present invention also provides methods of using the microneedles to delivery nanoparticles.

Thus according to another aspect, the present invention provides a method for delivering at least one nanoparticle(s) to a subject, wherein the delivery includes the steps of contacting a least an area of the subject with at least one microneedle associated with at least one nanoparticle, wherein at least one nanoparticle is delivered to the subject.

### BRIEF DESCRIPTION OF THE DRAWINGS

FIG. 1 shows a plan view of the needle cross-sections.

FIG. 2 shows a top view of PDMS microneedles with dye molecules added to colour the patches and microneedle.

FIG. 3 shows a side view of the crosses shown in FIG. 2.

FIG. 4 shows a side view of a microneedle array, needles are 20 μm diameter at the base and are on a 50 μm pitch.

FIG. 5 shows a top view of a sheet of multiple microneedle array patches.

FIG. 6 shows a magnified side view of one section of array patch shown in FIG. 5.

FIG. 7 shows a schematic flowchart of a process for forming nanopore(s) on the surface of a microneedle.

FIG. 8 shows a fluorescent image of an array of circular microneedles showing the coverage of the quantum dot coating.

FIG. 9 shows a fluorescent image of an array of cross shaped microneedles showing the coverage of the quantum dot coating.

FIG. 10 shows a scanning electron micrograph (SEM) image of insulin nanoparticles on PLGA microneedles.

FIG. 11 shows an SEM image of a microneedle array coated with insulin nanonpaticles.

FIG. 12 shows a confocal microscopy fluorescent image of a patch of skin removed from a hairless mouse.

FIG. 13 shows a confocal microscopy fluorescent image to a total depth of approximately 60 μm.

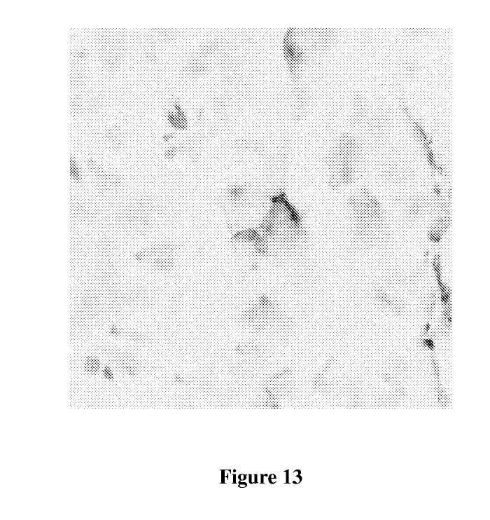

### DETAILED DESCRIPTION OF THE EMBODIMENTS

The devices disclosed herein are useful in transport of agent into or across biological barriers including the skin (or parts thereof); the blood-brain barrier; mucosal tissue (e.g., oral, nasal, ocular, vaginal, urethral, gastrointestinal, respiratory); blood vessels; lymphatic vessels; or cell membranes (e.g., for the introduction of material into the interior of a cell or cells). The biological barriers can be in humans or other types of animals, as well as in plants, insects, or other organisms, including bacteria, yeast, fungi, and embryos.

The microneedle devices can be applied to tissue internally with the aid of a catheter or laparoscope. For certain applications, such as for drug delivery to an internal tissue, the devices can be surgically implanted.

The present invention provides agents which can be a protein, peptide, cell homogenate, whole organism or glycoprotein effective as a sensing agent or protective agent.

The present invention also provides a presentation configuration of the agent in which for sensing, single molecules, multimers, aggregates, or multimer through nanoparticle anchoring may be used; whereas, for delivery (vaccination) the configuration of the biological molecule may also comprise: single molecules, multimers, aggregates, or multimers through nanoparticle anchoring.

Nanoparticle anchoring can be through nanoparticles of gold, silver, titanium, agarose, proteins, dendrimers, proteins or polymers. The preferred option is the multimeric nanoparticle presentation.

The present invention also has applications in the food industry for quality detection and for one or more infective agent(s), the infective agent can be a microorganism. The microorganism can be selected from one or more of the group comprising a virus, bacteria, protozoa and/or fungus.

The inventors have unexpectedly discovered that a novel delivery structure and composition, as well as the composition and configuration of the biological reagent for delivery and methods for their production. By forming the agents for delivery in the presence of removable and/or degradable nanoparticles of different composition to the composition of the delivery molecules, the nanostructured molecules incorporate a nanoporous structure capable of holding large and small molecules and nanoparticles-anchored biological molecules for delivery as vaccines and therapeutics.

It is also recognised that a number of novel polymer systems which when subjected to certain stresses change composition to have a nanoparticular structure which is different to the surrounding polymer, and such polymers can have application with their improved solubility (degradation properties) for the delivery of reagents from polymer array patches.

The aforementioned polyvalent nanoparticular vaccination particles can be released from polymer patches with penetration to the interstitial layer in live tissue

The aforementioned polyvalent nanoparticular sensing agents can be retained on the surface of the polymer patches with conducting properties for signal transduction.

The inventors have surprisingly found that the identical polymer is used for presenting (delivery/anchored sensing) the nanostructured molecule(s), and also unexpectedly, a polymer which although biocompatible is preferably not biodegradeable has advantages of speed of molecule delivery not requiring the lengthy time dependent degradation. In the aspect of the invention that has application to delivery for vaccination through the stratum corneum, resident time in this layer is of the order of two weeks.

In a further aspect of the present invention there is provided a process for delivering molecule(s) precisely to the appropriate depth using the microneedle arrays having nanostructured delivery molecules.

Construction of the device and control of structure of the polymer, by embedding nanoparticle-sized materials with properties to allow dissolution of the nanoparticles to create a mesoporous structure with nanoporous cavities for holding reagents or nanoparticle structured reagents. to be delivered by the array patch structure.

Both hollow and solid penetrator (solid needle) arrays are constructed with any of a range of sizes between 20 μm and 250 μm but the preferred sizes (lengths) are 25 μm and 150 μm.

The dimensions of the whole array could be in the order of 1 cm square or with a diameter of 1 cm. However, the size of the array patch would be based on the amount of material to be delivered and the needle density packing on the patches.

The microneedles are preferred to be in an array format, but could be randomly arranged. The arrangement of the microneedles may be a result of the method used in manufacture.

The microneedles may be arranged so that more than one reagent can be coated and delivered from the one array.

A polymer which when subjected to certain stresses change composition to have a nanoparticle structure which is different to the surrounding polymer, and such polymers can have application with their improved solubility (degradation properties) for the delivery of reagents from polymer array patches.

A polymer that contains a nanoparticle that can be selectively removed to produce nanosized pores or cavities on the microneedle surface.

The microneedle array patches of the present also provide applications for the treatment and prevention of human diseases. Preventative vaccination of a wide variety of human disease states can be achieved, for example, the present microneedle arrays can be used to vaccinate against any one or more of the disease states selected from the group comprising infectious diseases (including but not limited to meningococcal disease and tuberculosis) and autoimmune diseases (including but not limited to multiple sclerosis and rheumatoid arthritis).

As used herein, the term “nanoparticle”, is intended to include particles that range in size from about 1 nm to about 1000 nm. Preferably, the nanoparticles are in the range from about 50 nm to about 500 nm.

As used herein, the term “fabric”, is intended to describe the material which the particle is composed of.

As used herein, the term “biocompatible”, is intended to describe molecules that are not toxic to cells. Compounds are “biocompatible” if their addition to cells in vitro results in less than or equal to 20% cell death and do not induce inflammation or other such adverse effects in vivo.

As used herein, “associated” includes physical, chemical, and physiochemical attachment.

As used herein, “biodegradable” includes compounds are those that, when introduced into cells, are broken down by the cellular machinery into components that the cells can either reuse or dispose of without significant toxic effect on the cells (i.e., fewer than about 20% of the cells are killed).

The agent that can be delivered by use of the present invention includes any therapeutic substance which possesses desirable therapeutic characteristics. These agents can be selected from any one or more of the group comprising: thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroid or non-steroidal anti-inflammatory agents, immunosuppressive agents, growth hormone antagonists, growth factors, dopamine agonists, radiotherapeutic agents, peptides, proteins, enzymes, extracellular matrix components, ACE inhibitors, free radical scavengers, chelators, antioxidants, anti polymerases, antiviral agents, photodynamic therapy agents, and gene therapy agents.

In particular, the therapeutic substance can be selected from any one or more of the group comprising Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Imiquimod, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or syntheic glycoaminoglycans, M-CSF, metoclopramide, Midazolam, Monoclonal antibodies, Peglyated antibodies, PEGylated proteins or any proteins modified with hydrophilic or hydrophobic polymers or additional functional groups, Fusion proteins, Single chain antibody fragments or the same with any combination of attached proteins, macromolecules, or additional functional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF-, and TNF-antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, anti-depressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

### Johne's Disease

Paratuberculosis (Johne's disease) is a chronic, progressive enteric disease of ruminants caused by infection with Mycobacterium paratuberculosis. The disease signs of infected animals include weight loss, diarrhea, and decreased milk production in cows. Herd prevalence of Johne's disease is estimated to be 22-40% and the economic impact of this disease on the dairy industry was estimated to be over $200 million per year in 1996. In addition, M. paratuberculosis has been implicated as a causative factor in Crohn's disease, a chronic inflammatory bowel disease of human beings, which has served as a further impetus to control this disease in our national cattle industry. The treatment and prevention of Johne's disease has become a high priority disease in the cattle industry.

The membrane protein p34, SEQ ID No 1A, elicits the predominant humoral response against M. paratuberculosis and within the published sequence antigenic peptide epitopes have been identified, which include but are not limited to:

| | |
|---|---|
| NVESQPGGQPNT | (SEQ ID NO: 1) |
| QYTDHHS SLLGP | (SEQ ID NO: 2) |
| LYRP SD S SLAGP | (SEQ ID NO: 3) |

See for example, Ostrowski, M et al. (2003) Scandinavian Journal of Immunology, 58, 511-521.

Peptide regions on other potential antigens can also be used in the device which can include the antigens described in: Alkyl Hydroperoxide Reductases C and D Are Major Antigens Constitutively Expressed by Mycobacterium avium subsp. paratuberculosis. Olsen, et al. (2000) Infection and Immunity, 68(2), 801-808. Two proteins p11 and p20 have been identified as potential antigens for use in vaccination.

Thus suitably nano-structured vaccinations for Mycobacterium infection for diseases such as Johnes disease can be made and delivered according to the methods and devices of the current invention.

### Bovine Mastitis

Bovine mastitis is a serious problem, common in both lactating dairy-type and beef-type animals. The management of this disease is practiced mostly on the dairy-type animal where daily udder handling is required. Mechanical milking machines may have caused an increased incidence of mastitis; the true origins of the disease remain unknown. Bacterial organisms identified from affected glands are varied; however, the species of Streptococcus and Staphlococcus are most commonly isolated.

Purified proteins which act as antigens to Bovine mastitis have also be described and are incorporated by reference; Immunisation of dairy cattle with recombinant Streptococcus uberis GapC or a chimeric CAMP antigen confers protection against heterologous bacterial challenge. Fontaine et al. (2002) Vaccine, 2278-2286. It would be expected that specific peptide epitopes from these proteins would be antigenic.

PauA protein has been successfully used to vaccinate cattle to prevent mastitis caused by challenge infection with S. uberis (Leigh, J. A. 1999. “Streptococcus uberis: a permanent barrier to the control of bovine mastitis?” Vet. J. 157:225-238). Vaccinated, protected cattle generated serum antibody responses that inhibited plasminogen activation by PauA., S. uberis PauA protein sequence:

(SEQ ID NO: 4)
MKKWFLILMLLGIFGCATQPSKVAAITGYDSDYYARYIDPDENKIT
FAINVDGFVEGSNQEILIRGIHHVLTDQNQKIVTKAELLDAIRHQM
VLLQLDYSYELVDFAPDAQLLTQDRRLLFANQNFEESVSLEDTIQE
YLLKGHVILRKRVEEPITHPTETANIEYKVQFATKDGEFHPLPIFV
DYGEKHIGEKLTSDEFRKIAEEKLLQLYPDYMIDQKEYTIIKHNSL
GQLPRYYSYQDHFSYEIQDRQRIMAKDPKSGKELGETQSIDNVFEK
YLITKKSYKP

Epitope region peptides selected from this protein useful as vaccines candidates when presented in the appropriate nanoparticle form: including but not restricted to

| | |
|---|---|
| ILIRGIHHVL | (SEQ ID NO: 5) |
| IRHQMVLLQL | (SEQ ID NO: 6) |

As well as the whole or selected fragments of the protein sequence above.

### Meningococcal Disease

Omp85 proteins of Neisseria gonorrhoeae and N. meningitides and peptide sequences derived therefrom can be used as vaccines against the organisms causing meningococcal disease when presented in nanoparticle form, or variants according to US 2005074458, which is herein incorporated by reference.

And the gonococcal and opacity proteins according to EPO273116, including but not restricted to:

(SEQ ID NO: 7)
GRGPYVQADLAYAYEHITHDYP
(SEQ ID NO: 8)
STVSDYFRNIRTHSIBPRVSVGYDFGGWRIAADYARYRKWNDNKYSV

and their variants.

### Hepatitis C Virus

Fragments of the core protein used for in vitro immunisation can include but not be limited to:

| | |
|---|---|
| QDVKFPGGGVYLLPRRGPRL | (SEQ ID NO: 9) |
| RRGPRLGVRATRKTSERSQPRGRRQ | (SEQ ID NO: 10) |
| PGYPWPLYGNEGCGWAGWLLSPRGS | (SEQ ID NO: 11) |

These can be used in conjunction with or without Toll receptors and or lipoproteins as indicated by the following reference:

Cell activation by synthetic lipopeptides of the hepatitis C virus (HCV)—core protein is mediated by toll like receptors (TLRs) 2 and 4.

### Liver Fluke

Liver flukes (Fasciola spp.) infect a wide range of animals, including humans. The disease that is caused is termed Fasciolosis. As with most parasitic diseases, there is a complex life cycle.

Economically, sheep and cattle are of primary importance. Infection with liver fluke leads to decreased production due to poor energy conversion (meat and milk in cattle, meat and wool in sheep) and can lead to mortality (particularly in sheep).

Vaccines targeting liver fluke have been investigated for many years, with most subunit vaccines centered on Glutathione-S-transferase (GST), cathepsin L (catL) and fatty acid binding proteins (FABP). Attenuated vaccines, created by the irradiation of metacercariae, are very effective, however this method of vaccination is not commercially viable. Therefore, subunit vaccine candidates have been considered. DNA vaccines have been assessed and recombinant proteins such as cathepsin B been cloned and analysed. Antigens have been cloned and the use of cathepsin L proteases as vaccines described, see for example U.S. Pat. Nos. 6,623,735 and 20050208063, which is herein incorporated by reference.

The N-terminal sequences of the proteases to be used for in vitro immunisation can include but not be limited to:

| | |
|---|---|
| AVPDKIDPRBSG | (SEQ ID NO: 12) |

These can be incorporated into a nanoparticle(s) or can be formed as a nanoparticle.

### Injectable Nanoparticles

An injectable nanoparticle can be prepared that includes a substance to be delivered and a nanoparticular polymer that is covalently bound to the molecule(s), wherein the nanoparticle is prepared in such a manner that the delivery molecule(s) is on the outside surface of the particle. Injectable nano-structured molecule(s) with for example, antibody or antibody fragments on their surfaces can be used to target specific cells or organs as desired for the selective dosing of drugs.

The molecule for delivery can be covalently bound to the nanoparticular polymer by reaction with a terminal functional group, such as the hydroxyl group of a poly(alkylene glycol) nanoparticle by any method known to those skilled in the art. For example, the hydroxyl group can be reacted with a terminal carboxyl group or terminal amino group on the molecule or antibody or antibody fragment, to form an ester or amide linkage, respectively. Alternatively, the molecule can be linked to the poly(alkylene glycol) through a difunctional spacing group such as a diamine or a dicarboxylic acid, including but not limited to sebacic acid, adipic acid, isophthalic acid, terephthalic acid, fumaric acid, dodecanedicarboxylic acid, azeleic acid, pimelic acid, suberic acid (octanedioic acid), itaconic acid, biphenyl-4,4′-dicarboxylic acid, benzophenone-4,4′-dicarboxylic acid, and p-carboxyphenoxyalkanoic acid.

In this embodiment, the spacing group is reacted with the hydroxyl group on the poly(alkylene glycol), and then reacted with the molecule(s). Alternatively, the spacing group can be reacted with the molecule, such as an antibody or antibody fragment, and then reacted with the hydroxyl group on the poly(alkylene glycol). The reaction should by accomplished under conditions that will not adversely affect the biological activity of the molecule being covalently attached to the nanoparticle. For example, conditions should be avoided that cause the denaturation of proteins or peptides, such as high temperature, certain organic solvents and high ionic strength solutions, when binding a protein to the particle. For example, organic solvents can be eliminated from the reaction system and a water-soluble coupling reagent such as EDC used instead.

According to another embodiment, the agent to be delivered can be incorporated into the polymer at the time of nanoparticle formation. The substances to be incorporated should not chemically interact with the polymer during fabrication, or during the release process. Additives such as inorganic salts, BSA (bovine serum albumin), and inert organic compounds can be used to alter the profile of substance release, as known to those skilled in the art. Biologically-labile materials, for example, procaryotic or eucaryotic cells, such as bacteria, yeast, or mammalian cells, including human cells, or components thereof, such as cell walls, or conjugates of cellular can also be included in the particle.

Injectable particles prepared according to this process can be used to deliver drugs such as non-steroidal anti-inflammatory compounds, anaesthetics, chemotherapeutic agents, immunotoxins, immunosuppressive agents, steroids, antibiotics, antivirals, antifungals, and steroidal anti-inflammatories, anticoagulants. For example, hydrophobic drugs such as lidocaine or tetracaine can be entrapped into the injectable particles and are released over several hours. Loadings in the nanoparticles as high as 40% (by weight) can be achieved. Hydrophobic materials are more difficult to encapsulate, and in general, the loading efficiency is decreased over that of a hydrophilic material.

In one embodiment, an antigen is incorporated into the nanoparticle, alternatively, the antigen can compose the entire nanoparticle. The term antigen includes any chemical structure that stimulates the formation of antibody or elicits a cell-mediated humoral response, including but not limited to protein, polysaccharide, nucleoprotein, lipoprotein, synthetic polypeptide, or a small molecule (hapten) linked to a protein carrier. The antigen can be administered together with an adjuvant as desired. Examples of suitable adjuvants include synthetic glycopeptide, muramyl dipeptide. Other adjuvants include killed Bordetella pertussis, the liposaccaride of Gram-negative bacteria, and large polymeric anions such as dextran sulfate. A polymer, such as a polyelectrolyte, can also be selected for fabrication of the nanoparticle that provides adjuvant activity.

Specific antigens that can be loaded into the nanoparticles described herein include, but are not limited to, attenuated or killed viruses, toxoids, polysaccharides, cell wall and surface or coat proteins of viruses and bacteria. These can also be used in combination with conjugates, adjuvants, or other antigens. For example, Haemophilus influenzae in the form of purified capsular polysaccharide (Hib) can be used alone or as a conjugate with diptheria toxoid. Examples of organisms from which these antigens are derived include poliovirus, rotavirus, hepatitis A, B, and C, influenza, rabies, HIV, measles, mumps, rubella, Bordetella pertussus, Streptococcus pneumoniae, Clostridium diptheria, C. tetani, Vibrio Cholera, Salmonella spp., Neisseria spp., and Shigella spp.

The nanoparticle should contain the substance to be delivered in an amount sufficient to deliver to a patient a therapeutically effective amount of compound, without causing serious toxic effects in the patient treated. The desired concentration of active compound in the nanoparticle will depend on absorption, inactivation, and excretion rates of the drug as well as the delivery rate of the compound from the nanoparticle. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions.

The present invention will now be more fully described with reference to the accompanying examples. It should be understood, however, that the description following is illustrative only and should not be taken in any way as a restriction on the generality of the invention described above.

### Example 1

### Mould Formation Using a Polycarbonate Sheet

### Laser Ablation

A polycarbonate sheet was laser ablated using an excimer laser beam. The needle cross-section is determined by the shape of the aperture that the laser beam passes through prior to irradiating the polycarbonate workpiece. This process known as excimer laser photolithographic ablation, uses an imaging projection lens to form the desired shapes. The depth of laser ablation, and hence the maximum height of the cast material is determined by a computer program operating the excimer micromachining system.

Using excimer laser ablation of a polycarbonate sheet, a series of moulds for a microneedle arrays were fabricated with eleven different shapes and heights in the ranges of 20 μm to 200 μm.

Moulds were fabricated for a number of different microneedle shapes including square, circular, oval, cross needle, triangular, chevron, jagged chevron and half moon.

In addition to the shape of the microneedles, the density, depth and pitch of the microneedle were varied. For example, the laser ablation process was used to create moulds for two dense arrays:
a) 50 μm diameter shapes on a 50 μm pitch approx 100 μm high.
b) 100 μm diameter shapes on a 100 μm pitch approx 100 μm high

The moulds were evaluated to determine their suitability for fabrication process with a variety of techniques including optical microscopy, laser scanning confocal microscopy and scanning electron microscopy.

It has been our experience that good perforation structures are usually complex in cross section, and not normally simple conical protrusions. Hence shapes were chosen that contain edge features and symmetry that, lead to improved performance for perforation.

### Example 2

### Fabrication of Microneedle Arrays

Initial moulding trials were conducted with materials with two different viscosities. The most viscous material had a putty-like consistency, the second had a honey-like viscosity. These materials were applied to the polycarbonate moulds and pressure was applied via a glass tile to ensure the indentations were filled. To aid in the removal of gas bubbles in the moulds, a vacuum was applied to the moulded materials. The material was hardened by curing the polymer/polymer precursor using a sixty-second exposure to light from a handheld blue LED source through the glass tile.

Demoulding was a simple process, relying on the material's tendency to adhere more to the backing glass tile than to the polycarbonate mould. The moulds were made of polycarbonate sheet 250 to 500 μm thick and were more flexible than the glass tile. Hence the moulded material could be “peeled” from the slightly more flexible mould. The resultant structures were examined under an optical microscope. Some of the structures were measured using a laser scanning confocal microscope or imaged using a scanning electron microscope.

### Results

The second honey-like material filled the mould, and the air bubbles formed in the needle recesses of the mould and were removed through the application of a vacuum. Many of the structures demoulded satisfactorily and the mould was made usable for further trials with a combination of liquid and sonication cleaning.

A silicone release agent was applied to the polycarbonate to assist in demoulding, alternatively, materials such as PEEK or silicone elastomers could be used as the female moulds.

### Example 3

### Fabrication of Various Microneedle Arrays

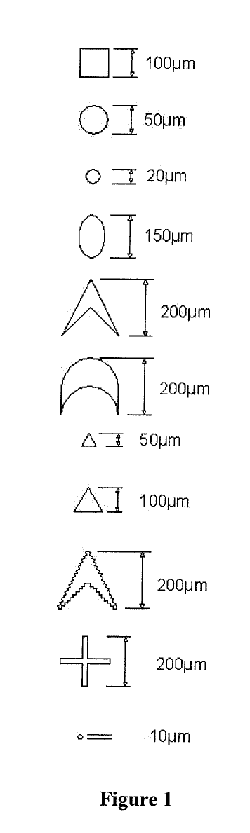

A number of microneedle arrays were fabricated with varying shapes, length, aspect ratios and needle densities. The various shapes are shown in FIG. 1.

### i) Cross-Shaped Needle Approximately 170 μm High

The cross-shaped needle moulds filled well with polymer, including the point at the intersection of the cross that is formed as a result of the ablation process. The combination of the relatively large side arms and the fine feature at the apex produces a robust structure with good mechanical properties.

### ii) Circular Microneedle 50 μm in Diameter

The circular microneedle approximately 140 μm high with an aspect ratio of about 3 was produced.

iii) Triangular Microneedle 50 μm on a Side

A triangular microneedle which is approximately 100 μm high and has an aspect ratio of about 2 was prepared. The smooth apex of the shape is due to the polymer moulding material and has not fully reproduced the fine texture of the ablated mould.

### iv) Circular Microneedles

An array patches with circular microneedle 20 μm in diameter and 50 μm high and 100 μm in diameter at 100 μm pitch, approximately 100 μm high were produced

v) Oval, Chevron, Jagged Chevron, Triangle, Half moon and Diamond Shapes

A variety of different shaped needle profiles were produced to investigate the effect on skin perforation on the shape of the microneedle.

### Example 4

### Fabrication of Array Patches with Coloured Spikes and Crosses

Array patches with a series of coloured spikes and crosses were constructed from polydimethylsiloxane (PDMS), a clear elastomer material by excimer laser machining 2 moulds in polycarbonate with four patches of 10 mm×10 mm each, with female features of tapering circular structures, and crosses. The pitch and depths of the structures were varied. Clear and coloured PDMS was cast from these features.

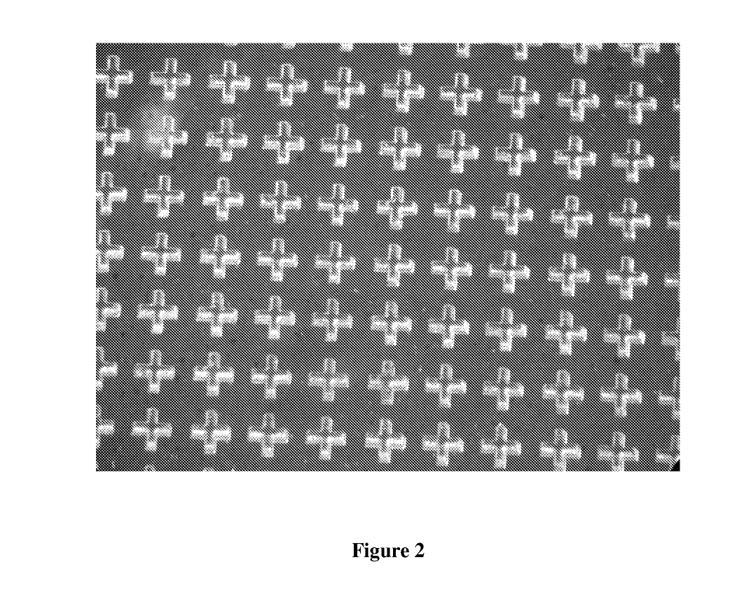

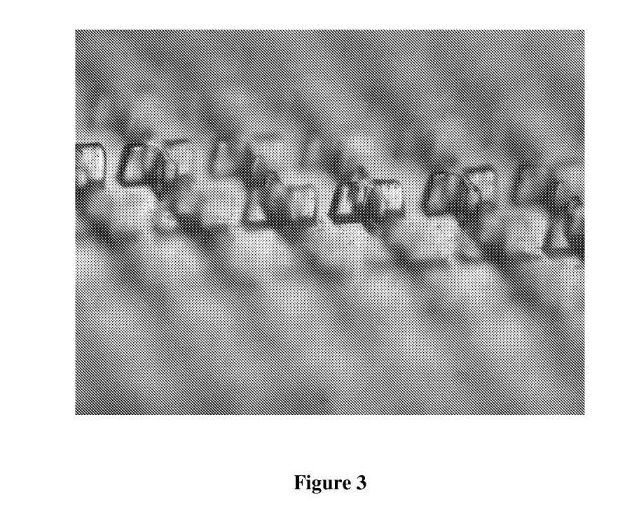

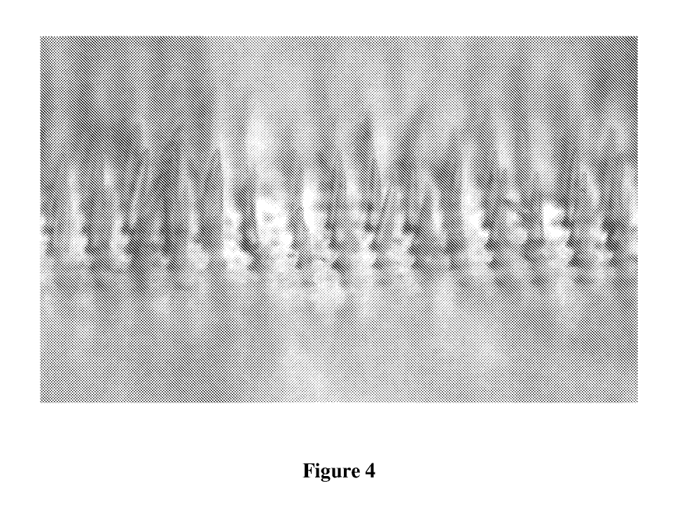

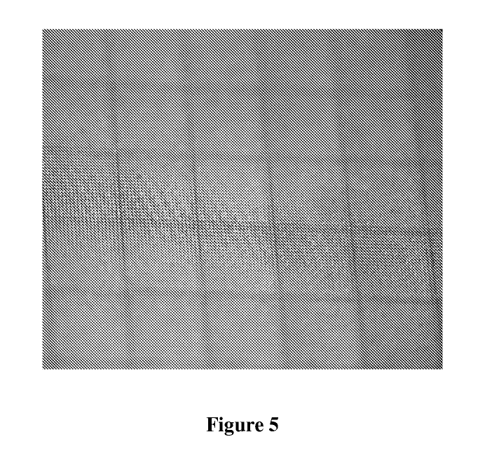

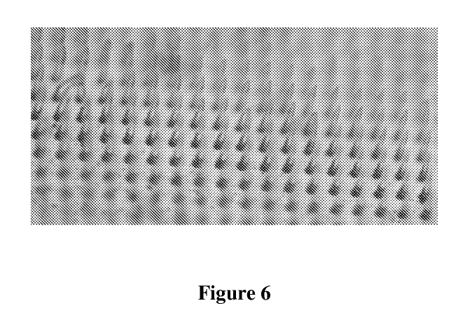

Initial moulding trials were conducted with standard PDMS supplied by DUPONT. This is a two part formulation, with 10% accelerator added to cause the material to set. The mixture was placed in a vacuum chamber to speed up outgassing prior to moulding to prevent bubble formation during curing. FIG. 2 shows a top view of a fabricated PDMS cross shaped microneedles and FIG. 3 shows the side view of the fabricated cross shaped microneedles. FIGS. 4, 5 and 6 show various microneedle arrays prepared according to the described methods.

Aqueous based colouring was added to the PDMS prior to casting; adding larger quantities of colouring intensified the colour, additional curing accelerator was added to compensate for the volume of aqueous colouring added.

The material was hardened by curing the moulded material by placing in a 45° C. oven for several hours. Curing rates were significantly slower for the coloured material.

Somewhat surprisingly demoulding the aqueous coloured material was more successful than the non-coloured material. This could be due to a range of effects such as increased curing accelerator, casting thicker pieces that tended to hold onto the needles more effectively during demoulding, or perhaps some inhibition of adhesion between PDMS and polycarbonate as a result of the aqueous additive.

### Example 5

### Post Curing Modification of the Microneedle Arrays

The microneedles produced by the method of Example 3 can be coated with a layer of a biocompatible electrically conducting polymer to modify the delivery characteristics of the microneedle. Thus to assist in the delivery of certain types of molecules, a polyaniline coating can be applied to the solid polymeric microneedle after demoulding. The conducting polymer can be applied using techniques known in the art, including electrodeposition.

During the electrodeposition phase (including polymerisation) biological reagents (for vaccines, drug delivery etc) can be included in the conductive polymer. The conductive polymer can be polymerised (electrodeposited) under conditions in such a way as that the electrodeposited polymer surface has characteristics that enable the diffusion of the biological reagent out into the surrounding environment (skin) in order for the biological reagent to be functional for its purpose.

A number of different thickness coatings can be applied depending on the desired application, ranging from 20 nm to 20 μm can be produced.

In another experiment, polyaniline and polypyrrole can be codeposited electrochemically on microneedles made from conductive materials under potentiostatic or galvanostatic conditions. Electropolymerisation can be carried out by varying the applied potential and the feed ratio of monomers. Formation of polyaniline-polypyrrole composite coatings can be confirmed by the presence of characteristic peaks for polyaniline and polypyrrole in the infrared spectra. Composite coatings composed of polyaniline and polypyrrole can be formed at applied potentials of <1.0 V. Polypyrrole is preferentially formed at 1.5 V.

Methods of electrodeposition have been described previously and include Adeloju, S. B. and Shaw, S. J., (1993) “Polypyrrole-based potentiometric biosensor for urea” Analytica Cimica Actica, 281, page 611-620; Adeloju S. B. and Lawal, A., (2005) Intern. J. Anal. Chem., 85, page 771-780, based on their use as a sensor. We have surprising found that the techniques can be applied to incorporating proteins and peptides into a polymer layer for delivery of the proteins and peptides as therapeutics such as peptide and protein antigens (for vaccines), hormones (erythropoietin, parathyroid hormone) and drugs (insulin).

### Example 6

### Nanoparticles for Delivery

The nanoparticles can be formed from metals (gold silver) light metals, polymer material by any of the standard techniques (U.S. Pat. No. 6,908,496 to Halas et al.; U.S. Pat. No. 6,906,339 to Dutta; U.S. Pat. No. 6,855,426 to Yadav; U.S. Pat. No. 6,893,493 to Cho et al.). The surface of the nanoparticles can be functionalised to anchor/immobilise (multimerise) the biological reagents for improved immunisation efficiency.

Other non-limiting examples of methods for nanoparticle formation include:

Cao L, Zhu T and Liu Z (2005) “Formation mechanism of nonspherical gold nanoparticles during seeding growth: role of anion adsorption and reduction rate.” Journal of Colloid Interface Science, July 11.

Bilati U, Alleman E and Doelker E. (2005) “Poly (D,L-lactide-co-glycolide) protein-loaded nanoparticles prepared by the double emulsion method—processing and formulation issues for enhanced trapment efficiency.” Journal of Microencapsulation, 22(2), 205-214.

Rolland J P, Maynor B W, Euliss L E, Exner A E, Denison G M and Desimone J M (2005) “Direct fabrication and harvesting of monodisperse, shape specific nanobiomaterials.” Journal of the American Chemical Society, 127(28), 10096-100.

The biological agents can be immobilized on the surface of a nanoparticle or integrally incorporated inside the nanoparticle during fabrication. The delivery agent may also be directly manufactured or naturally present in a nanoparticulate form.

The biological agents Insulin and ovalbumin were structured as nanoparticles using supercritical fluid technology, to produce nanoparticles of dimensions 50-300 nm. The insulin nanoparticles were suspended in a solvent (ethanol) and attached to the surface of the microneedles. Insulin and ovalbumin attached to microneedles are each being delivered separately across the stratum corneum and the response to the delivery of insulin can be measured.

Erythropoietin is a glycoprotein hormone produced in the liver during foetal life and the kidneys of adults and is involved in the maturation of erythroid progenitor cells into erythrocytes. There are several human conditions and treatments for cancer which result in low levels of circulating red blood cells and therefore administration of erythropoietin is desirable. Erythropoietin can be nanostructured by supercritical fluid technology and attached to microneedles for delivery by microneedle array, and delivery efficiency can be measured by physiological effects on red cell numbers in mice (including flow cytometry).

### Example 7

### Nanoparticles for Creating Nanopores in the Array Patch Microneedles

The surface of a polymeric microneedle array can be nano-structured during fabrication by lining the microneedle mould with nanoparticles which can be selectively removed. The microneedles can then be cast, hardened and demoulded to produce microneedles with nanoparticles embedded on the surface of the microneedles.

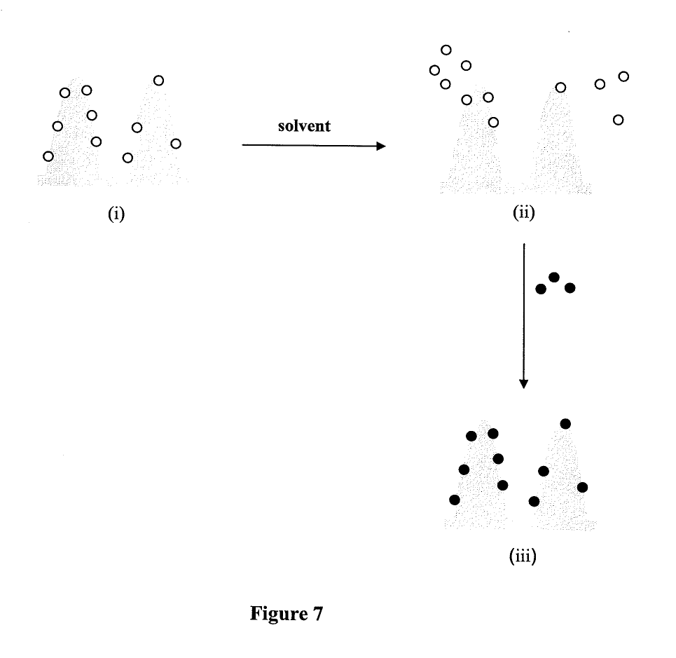

The embedded nanoparticles can then be removed, for example by dissolution or leeching techniques, to yield a microneedle that has nano-sized pores or cavities on their surface. The delivery agent molecules or nanoparticles can then be associated with the introduced pores by non-covalent interactions or covalent bonds. Referring to the process shown in FIG. 7, the method includes the steps of:

(i) Soluble “template” nanoparticles incorporated into microneedles during patch manufacture;

ii) Template nanoparticles removed with solvent leaving recesses over microneedle surface and then nano-structured reagent(s) are added to the solution;

iii) Nanostructured reagent(s) fits into recesses within needle structure to form the microneedles with the nanostructured reagents associated with the microneedles.

The moulded microneedle can alternatively be chemically treated with a solvent, chemical reagent, electrochemcial or physical treatment to induce surface cavity and/or nanopore formation.

### Example 8

### Microneedles Made from Electrically Conducting Polymers

A polyaniline microneedle array can be fabricated by electropolymerization of a monomer solution contained in a microneedle array mould under an applied potential. The progress of electropolymerisation can be monitored by weight gain analysis and infrared spectroscopy.

The nanoparticles can be added to the monomer solution prior to polymerization to form a microneedle array with the delivery molecule integrally incorporated into the needles, or the nanoparticles can be associated to the surface of the microneedles by a post demoulding step.

### Example 9

### Coating of Quantum Dots onto the Microneedle Arrays

To demonstrate the efficacy for the loading of patches with nanoparticles, a series of microneedle arrays was coated with Quantum Dots. Quantum Dots are semiconductor crystals typically between 1 and 10 nm in diameter and have unique properties between that of single molecules and bulk materials. Under the influence of an external electromagnetic radiation source, quantum dots can be made to fluoresce and therefore their position accurately determined using readily available optical techniques.

Circular microneedle array patches with both bullet and cross shaped needles were constructed in PLGA (Poly-DL-lactic glycolic acid, 0.8 cm in diameter with a 2 mm edge). The patches were coated with Quantum Dots by placing 100 μL of CdSe/ZnS Quantum Dots (200 picoMolar, Invitrogen Qtracker™ 655 nm) on top of the microneedles and air drying. The arrays were examined for fluorescence using confocal microscopy.

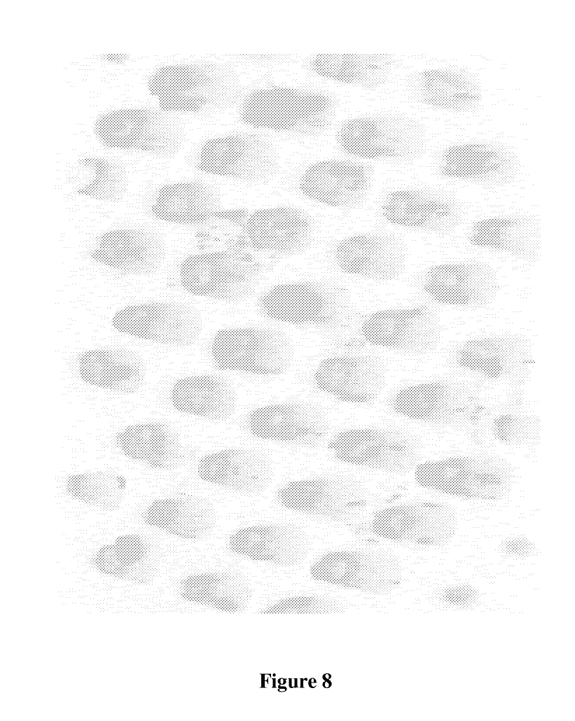

The arrays demonstrated red fluorescence on the both the bullet and cross shaped needles indicating coating by the Quantum Dots. As shown in FIGS. 7, coverage was shown at the tops over the needles and down the sides to the base. The cross shaped needles demonstrated more confluent coverage of quantum dots, as shown in FIG. 8.

The uptake of Quantum Dots by lymphocytes can be observed by in vitro studies on cultured cells and by in vivo studies on hairless mouse models.

### Example 10

### Coating of Insulin Nanoparticles onto the Microneedle Arrays

To demonstrate the efficacy for the loading of patches with nanoparticulate biological molecules, a series of microneedle array patches were coated with nanostructured insulin. Insulin can be nanostructured using various methods including super critical fluid technologies. The particle size of the insulin averaged 300 nm.

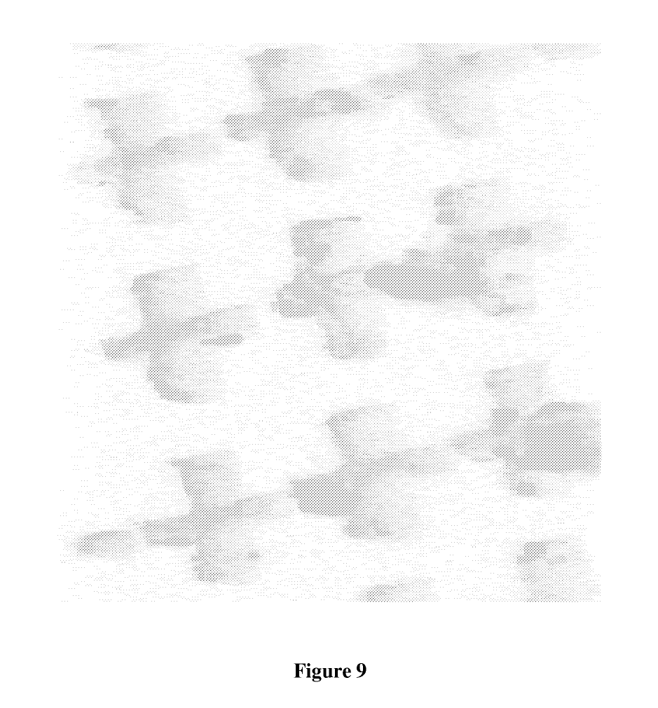

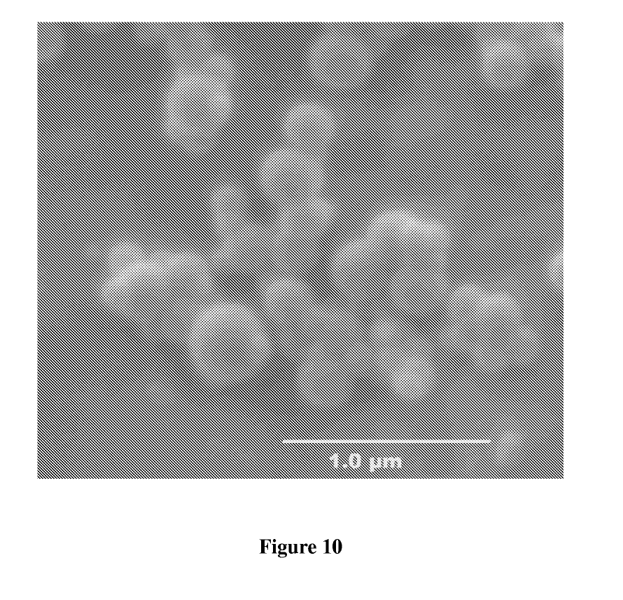

Circular PLGA patches in high density cross and needle shapes were coated with the nanostructured insulin by placing 100 μL of nanostructured insulin in iso-amyl alcohol (total 0.6 Units insulin/patch) on top of the patches and air drying. The patches were then examined for the presence of insulin using Field Emission Gun Scanning Electron Microscope (FEG-SEM), as shown in FIGS. 9 and 10.

The patches demonstrated the presence of nanostructured insulin both over the top surfaces of the microneedles and down the side edges of the needles. The density of the insulin nanoparticles on the cross shaped microneedles was much lower due to the higher surface area of the crosses compared to the bullets.

### Example 11

### Demonstration of Skin Penetration and Delivery of Quantum Dots

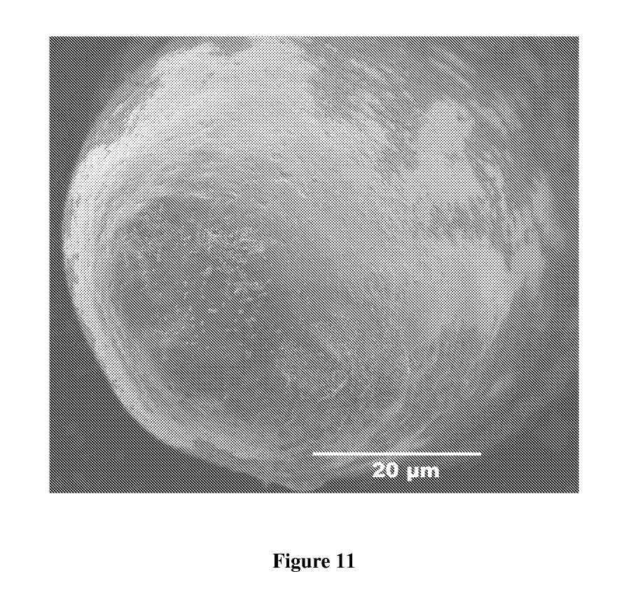

Bullet shaped patches were coated with Quantum dots by placing 100 μL of CdSe/ZnS Quantum dots (200 picoMolar in saline, Invitrogen Qtracker™ 655 nm) on top of the microneedles and air drying. The patches were applied to the rear flank of hairless mice by manually pressing. The patch was removed and the skin excised and examined for fluorescence using confocal microscopy, as shown in FIG. 11.

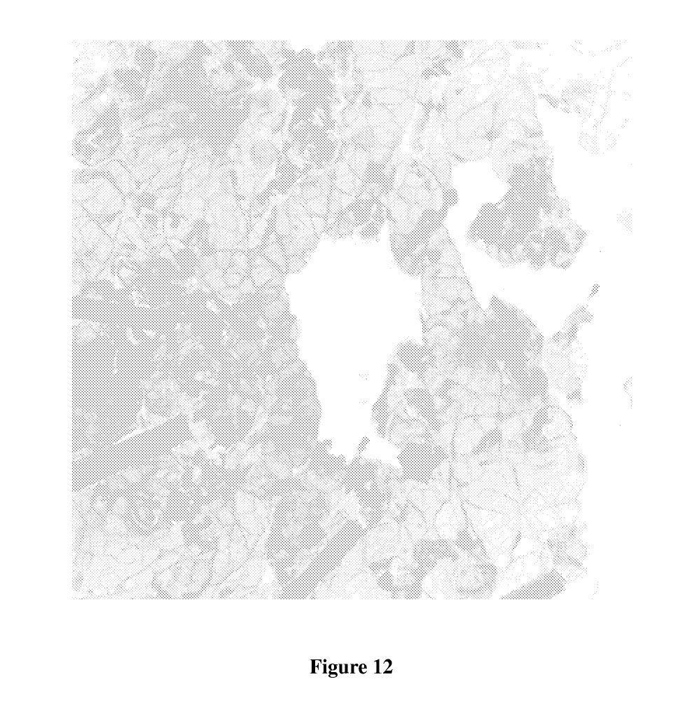

The skin demonstrated red fluorescence on the surface of the stratum corneum indicating deposition of the Quantum Dot present on the base of the array. Confocal imaging deeper into the epidermis indicated red fluorescence in the shape of a bullet demonstrating penetration of the microneedle to a total depth of approximately 60 μm, as shown in FIG. 12. This experiment demonstrates conclusively that the microneedle array can be used to deliver nanoparticles across stratum corneum layer of the dermis.

### Example 12

### Delivery of nanostructured insulin using microarray patches

### Preparation of Insulin Nanoparticles

Insulin was nanostructured using a supercritical fluid process. An average particle size of 300 nm was obtained. The insulin was suspended in various solvents including isopropanol, isoamyl ethanol, ethanol, methanol or other coatings onto the array.

For coating of the microarrays, insulin nanoparticles were suspended in solvent to a final concentration of 120 U/ml (4.32 mg/ml) and sonicated for 60 seconds to ensure complete dispersal throughout the suspension. The suspension was then applied to each microarray (6 U in 50 μl) and allowed to air dry.

For subcutaneous delivery in the control experiments, the solution used to coat the microarrays was diluted 1:300 in normal saline (final concentration of 0.4 U/ml).

### Blood Glucose Experiments

Hairless mice were anaesthetised with pentobarbitone (60 mg/kg, i.p.). Blood samples were obtained by tail laceration and blood glucose was measured using a commercial glucose-meter (Optimum™ Xceed™; Abbot Diagnostics). After obtaining two consecutive readings, mice were treated as indicated and blood glucose was recorded every 20 minutes for the remainder of the experiment. Mice were treated with either a positive control (insulin suspension, 1 U/kg, s.c.), insulin loaded microarrays (2 patches for each mouse, 6 U/patch), or negative control (12 U insulin applied directly to the skin without any microarray). Administration of the insulin via the microarray patch can be shown in the mouse by a change in the blood glucose levels.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A device suitable for delivering at least one nanoparticle comprising a microneedle having at least one nanoparticle associated with at least part of a surface of the microneedle and/or at least part of the fabric of the microneedle.

2. The device according to claim 1, wherein the device has at least two microneedles.

3. The device according to claim 1, wherein the device has at least two microneedles in a non-patterned arrangement, array or other such configuration.

4. The device according to claim 1, wherein the nanoparticle(s) is/are associated with at least a part of the external surface of the microneedle.

5. The device according to claim 1, wherein the nanoparticle(s) is/are associated with pores on the surface of the microneedles.

6. The device according to claim 1, wherein the nanoparticle(s) is/are associated with at least a part of the fabric of the microneedle.

7. The device according to claim 1, wherein the nanoparticle(s) is/are associated with all of the fabric of the microneedle.

8. The device according to claim 1, wherein the nanoparticle(s) is/are associated with internal pores in the fabric of the microneedle.

9. The device according to claim 1, wherein the association comprises a non-covalent interaction selected from any one or more of the group consisting of ionic bonds, hydrophobic interactions, hydrogen bonds, Van der Waals forces and Dipole-dipole bonds.

10. The device according to claim 1, wherein the association is via a covalent bond to a functional group on the microneedle.

11. The device according to claim 1, wherein the association is via a covalent bond to a functional group on the microneedle and the functional group(s) is/are selected from the group consisting of COOR, CONR.sub.2, NH.sub.2, SH, and OH, wherein R comprises a H, an organic chain, or an inorganic chain.

12. The device according to claim 1, wherein the microneedle(s) is/are fabricated from a porous or non-porous material selected from the group consisting of metals, natural or synthetic polymers, glasses, ceramics, and combinations of two or more thereof.

13. The device according to claim 1, wherein the microneedle(s) is/are fabricated from a polymer selected from the group consisting of: polyglycolic acid/polylactic acid, polycaprolactone, polyhydroxybutarate valerate, polyorthoester, and polyethylenoxide/polybutylene terepthalate, polyurethane, silicone polymers, and polyethylene terephthalate, polyamine plus dextran sulfate trilayer, high-molecular-weight poly-L-lactic acid, fibrin, methylmethacrylate (MMA) (hydrophobic, 70 mol %) and 2-hydroxyethyl methacrylate (HEMA) (hydrophilic 30 mol %), elastomeric poly(ester-amide)(co-PEA) polymers, polyetheretherketone, (Peek-Optima), biocompatible thermoplastic polymer; conducting polymers, polystyrene and combinations of two or more thereof.

14. The device according to claim 1, wherein the microneedle(s) includes a layer or coating on at least a part of the surface of the microneedle(s) of an electrically conductive material.

15. The device according to claim 1, wherein the microneedle(s) includes a layer or coating on at least a part of the surface of the microneedle(s) of an electrically conductive material selected from the group consisting of conducting polymers; conducting composite materials; doped polymers, conducting metallic materials and combinations of two or more thereof.

16. The device according to claim 1, wherein the microneedle(s) includes a layer or coating on at least a part of the surface of the microneedle(s) of an electrically conductive material selected from the group consisting of: (i) substituted or unsubstituted polymers comprising polyaniline, polypyrrole, polysilicones, or poly(3,4-ethylenedioxythiophene); (ii) polymer doped with carbon nanotubes; (iii) polymer doped with metal nanoparticles; and (iv) combinations of two or more thereof.

17. The device according to claim 1, wherein the microneedle(s) includes a layer or coating on at least a part of the surface of the microneedle(s) of an electrically conductive material and the thickness of the layer or coating is between about 20 nm to about 20 .mu.m.

18. The device according to claim 1, wherein the microneedle(s) includes a layer or coating on at least a part of the surface of the microneedle(s) of an electrically conductive material and wherein the electrically conductive material is layered or coated on the microneedle(s) by electrodeposition.

19. The device according to claim 1, wherein the nanoparticle(s) is/are delivered to an organism and the microneedle(s) is fabricated from a biocompatible material.

20. The device according to claim 1, wherein the microneedle(s) is/are non-biodegradable.

21. The device according to claim 1, wherein the or each microneedle is solid.

22. The device according to claim 1, wherein the nanoparticle(s) is/are an active agent.

23. The device according to claim 1, wherein the nanoparticle(s) is/are a carrier.

24. The device according to claim 1, wherein the nanoparticle is associated with an active agent.

25. The device according to claim 1, wherein the nanoparticle is associated with an active agent by covalent or non-covalent bonding.

26. The device according to claim 1, wherein the nanoparticle encapsulates an active agent.

27. The device according to claim 1, wherein the nanoparticle(s) is/are fabricated from a material selected from the group consisting of metals, semiconductors, inorganic or organic polymers, magnetic colloidal materials, and combinations of two or more thereof.

28. The device according to claim 1, wherein the nanoparticle(s) is/are fabricated from a metal selected from the group consisting of gold, silver, nickel, copper, titanium, platinum, palladium, oxides thereof, and combinations of two or more thereof.

29. The device according to claim 1, wherein the nanoparticle(s) is/are fabricated from a polymer is selected from the group consisting of a conducting polymer; a hydrogel; agarose; polyglycolic acid/polylactic acid; polycaprolactone; polyhydroxybutarate valerate; polyorthoester; polyethylenoxide/polybutylene terepthalate; polyurethane; polymeric silicon compounds; polyethylene terephthalate; polyamine plus dextran sulfate trilayer; high-molecular-weight poly-L-lactic acid; fibrin; copolymers of methylmethacrylate (MMA) and 2-hydroxyethyl methacrylate (HEMA), elastomeric poly(ester-amide)(co-PEA) polymers; n-butyl cyanoacrylate; polyetheretherketone; (Peek-Optima), polystyrene and combinations of two or more thereof.

30. The device according to claim 1, wherein the nanoparticle(s) is a biologically active agent.

31. The device according to claim 1, wherein the nanoparticle(s) is a therapeutic and/or a diagnostic agent.

32. The device according to claim 1, wherein the nanoparticle(s) is a therapeutic agent selected from the group consisting of peptides, proteins, carbohydrates, nucleic acid molecules, an oligonucleotide or a DNA or RNA fragment(s), lipids, organic molecules, biologically active inorganic molecules and combinations of two or more thereof.

33. The device according to claim 1, wherein the nanoparticle(s) is a vaccine.

34. The device according to claim 1, wherein the nanoparticle(s) is a vaccine selected from the group consisting of a vector containing a nucleic acid, oligonucleotide, gene for expression as a vaccine and combinations of two or more thereof.

35. The device according to claim 1, wherein the nanoparticle(s) is a vaccine selected from proteins or peptides as vaccines for diseases selected from the group consisting of Johnes disease, bovine mastitis, meningococcal disease and combinations of two or more thereof.

36. The device according to claim 1, wherein the nanoparticle(s) is a vaccine comprising a Johnes disease peptide selected from the group consisting of: TABLE-US-00011 NVESQPGGQPNE; (SEQ ID NO: 1) QYTDHHSSLLGP; (SEQ ID NO: 2) and LYRPSDSSLAGP. (SEQ ID NO: 3).

37. The device according to claim 1, wherein the nanoparticle(s) is a bovine mastitis disease peptide selected from the group consisting of: TABLE-US-00012 (SEQ ID NO: 4) MKKWFLILMLLGIFGCATQPSKVAAITGYDSDYYARYIDPDENKITFAIN VDGFVEGSNQEILIRGIHHVLTDQNQKIVTKAELLDAIRHQMVLLQLDYS YELVDFAPDAQLLTQDRRLLFANQNFEESVSLEDTIQEYLLKGHVILRKR VEEPITHPTETANIEYKVQFATKDGEFHPLPIFVDYGEKHIGEKLTSDEF RKIAEEKLLQLYPDYMIDQKEYTIIKHNSLGQLPRYYSYQDHFSYEIQDR QRIMAKDPKSGKELGETQSIDNVFEKYLITKKSYKP; (SEQ ID NO: 5) ILIRGIHHVL; and (SEQ ID NO: 6) IRHQMVLLQL.

38. The device according to claim 1, wherein the nanoparticle(s) is a detectable diagnostic agent.

39. The device according to claim 1, wherein the outer diameter of the microneedle(s) is/are between about 1.mu.m and about 100.mu.m.

40. The device according to claim 1, wherein the length of the microneedle(s) is/are between about 20.mu.m and 1 mm.

41. The device according to claim 1, wherein the length of the microneedle(s) is/are between about 20.mu.m and 250.mu.m.

42. The device according to claim 1, wherein the microneedle(s) is/are adapted to provide an insertion depth of less than about 100 to 150.mu.m.

43. The device according to claim 1, wherein the shape of the microneedle(s) tip is/are selected from the group consisting of square, circular, oval, cross needle, triangular, chevron, jagged chevron, half moon and diamond shaped.

44-65. (canceled)

66. A microneedle comprising a plurality of biodegradable nanoparticles, wherein the nanoparticles are removable and/or a degradable nanoparticles.

67. (canceled)


SEQUENCE LISTING (12 sequences)

SEQ ID NO: 1 - protein, 12 aa, Mycobacterium paratuberculosis
NVESQPGGQPNT

SEQ ID NO: 2 - protein, 12 aa, Mycobacterium paratuberculosis
QYTDHHSSLLGP

SEQ ID NO: 3 - protein, 12 aa, Mycobacterium paratuberculosis
LYRPSDSSLAGP

SEQ ID NO: 4 - protein, 286 aa, Streptococcus uberis
MKKWFLILMLLGIFGCATQPSKVAAITGYDSDYYARYIDPDENKITFAINVDGFVEGSNQEILIRGIHHVLTDQNQKIVTKAELLDAIRHQMVLLQLDYSYELVDFAPDAQLLTQDRRLLFANQNFEESVSLEDTIQEYLLKGHVILRKRVEEPITHPTETANIEYKVQFATKDGEFHPLPIFVDYGEKHIGEKLTSDEFRKIAEEKLLQLYPDYMIDQKEYTIIKHNSLGQLPRYYSYQDHFSYEIQDRQRIMAKDPKSGKELGETQSIDNVFEKYLITKKSYKP

SEQ ID NO: 5 - protein, 10 aa, Streptococcus uberis
ILIRGIHHVL

SEQ ID NO: 6 - protein, 10 aa, Streptococcus uberis
IRHQMVLLQL

SEQ ID NO: 7 - protein, 22 aa, Neisseria gonorrhoeae
GRGPYVQADLAYAYEHITHDYP

SEQ ID NO: 8 - protein, 47 aa, Neisseria gonorrhoeae
STVSDYFRNIRTHSIHPRVSVGYDFGGWRIAADYARYRKWNDNKYSV

SEQ ID NO: 9 - protein, 20 aa, Hepatitis C virus
QDVKFPGGGVYLLPRRGPRL

SEQ ID NO: 10 - protein, 25 aa, Hepatitis C virus
RRGPRLGVRATRKTSERSQPRGRRQ

SEQ ID NO: 11 - protein, 25 aa, Hepatitis C virus
PGYPWPLYGNEGCGWAGWLLSPRGS

SEQ ID NO: 12 - protein, 12 aa, Fasciola spp
AVPDKIDPRBSG